(19) United States
(12) Patent Application Publication
(10) Pub. No.: US 2024/0323524 A1
(43) Pub. Date: Sep. 26, 2024

(54) Optical coherence tomography image system and autofocus method thereof

(71) Applicant: Medimaging Integrated Solution, Inc., Hsinchu (TW)

(72) Inventors: Jia-Pu Syu, Hsinchu (TW); CHIEN-CHI HUANG, Hsinchu (TW); Yu-Tsung Lee, Hsinchu (TW); Hung-Chin Chen, Hsinchu (TW)

(21) Appl. No.: 18/615,710

(22) Filed: Mar. 25, 2024

(30) Foreign Application Priority Data
Mar. 26, 2023 (TW) 112111407

(51) Int. Cl. H04N 23/67; A61B 3/10; A61B 3/12; A61B 3/14; G06T 7/00

(52) U.S. Cl. CPC H04N 23/67; A61B 3/102; A61B 3/12; A61B 3/14; G06T 7/0014; G06T 2207/10101; G06T 2207/30041; G06T 2207/30204

(57) ABSTRACT

An optical coherence tomography image system and an autofocus method thereof are provided. The autofocus method captures a reference chart at a plurality of preset diopter positions, receives an interference signal generated by the reflection of a light beam projected onto an eyeball, and analyzes the reference chart and the interference signal to obtain an analysis value and a corresponding diopter. The method compares the plurality of analysis values to obtain one meeting the standard condition as the target analysis value. According to the target analysis value and the corresponding diopter, the focusing driving device is controlled to move to the position corresponding to the diopter to complete autofocusing. Therefore, the present invention can reduce the time of autofocusing, obtain a higher shooting success rate, provide better user experience, and use a lower-order computing device to reduce costs.

### BACKGROUND OF THE INVENTION

### 1. Field of the Invention

The present invention relates to an image system and a focusing method thereof, particularly to an optical coherence tomography image system and an autofocus method thereof.

### 2. Description of the Prior Art

For ophthalmologic diseases, such as maculopathy, diabetic macular edema and retinal vein occlusion, the current technology may use an optical coherence tomography (OCT) inspection device to effectively monitor the therapy effect. Further, the physicians may frequently use the OCT images to monitor the variation of the eyeball and adjust the dosage of medicine in real time. In the previous therapy method, the patients must be injected with a high dosage of medicine periodically. The OCT device enables the physicians to track the patients' condition, favoring lowering medical expense, exempting the patients from planless therapies. However, a hospital's OCT device is very expensive, and the operation thereof is complicated. Further, the patients must spend a lot of time in going to the hospitals for OCT inspection routinely. Therefore, OCT inspection would consume much time of patients and manpower of hospitals. The requirement for household OCT devices does exist currently. However, the overall cost of a household OCT device is still very high. If the household OCT device is intended to be popularized to homes, the price thereof should be lowered to such an extent that the ordinary people can afford it.

The conversion from primitive spectral signals to OCT images needs massive computation and signal procession, such as k-space resampling, Fourier Transform, and diversion compensation. The signal procession would increase the time of autofocusing and needs to use a high-performance computing device. If the household OCT device adopts a lower-performance computing device to reduce the fabrication cost, it would slow down the focusing process and increase scanning time. Thus, the user may feel less comfortable, and the user experience will be degraded. Besides, the failure rate of shooting may increase.

Accordingly, the present invention proposes an optical coherence tomography image system and an autofocus method thereof to overcome the problems of the conventional technologies.

### SUMMARY OF THE INVENTION

Considering the abovementioned conventional problems, the present invention proposes an optical coherence tomography image system and an autofocus method thereof, whereby to shorten autofocusing time, obtain higher shooting success rate, provide better user experience, and use a lower order computing device for reducing cost.

In order to achieve the abovementioned objectives, the present invention provides an autofocus method, which is applied to an optical coherence tomography image system, and which comprises a moving step, a signal receiving step, a signal analyzing step, a comparing step, and a focusing step.

In the moving step, move a focusing driving device within a preset diopter range to reach one of a plurality of preset diopter positions. In the signal receiving step, capture reference charts at the plurality of preset diopter positions and receive an interference signal generated by the reflection of a light beam projected on an eyeball.

In the signal analyzing step, analyze the reference charts and the interference signals to obtain the analysis values and the corresponding diopters. The moving step and the signal receiving step are performed repeatedly to obtain the reference charts and the interference signals respectively corresponding to the plurality of preset diopter positions. During the signal analyzing step, a plurality of reference charts and a plurality of interference signals are analyzed to obtain a plurality of corresponding analysis values and a plurality of corresponding diopters. In the comparing step, compare the plurality of analysis values to obtain from the plurality of analysis values one analysis value meeting a standard condition as a target analysis value.

In the focusing step, according to the target analysis value and the corresponding diopter, control a focusing driving device to move to the corresponding diopter position, whereby autofocusing is completed.

In one embodiment, before the moving step of the autofocusing method is started, a fundus camera is turned on to perform autofocusing in advance. The moving step is not undertaken unless the fundus camera has completed autofocusing. The reference chart is the ocular image of the eyeball. The signal receiving step further comprises a step: controlling the fundus camera to capture ocular images at the plurality of preset diopter positions.

The analysis value is a mark area or a mark intensity. The signal analyzing step further comprises a step: analyzing a light source mark of the light beam in the ocular image to obtain the mark area or mark intensity of the light source mark.

In the comparing step, the standard condition is a minimum value of a plurality of mark areas or a maximum value of a plurality of mark intensities.

The autofocus method of this embodiment is applicable to an OTC (optical coherence tomography) image system having a fundus camera, whereby to reduce image shooting time, achieve a higher shooting success rate, and provide better user experience.

In one embodiment, the reference chart is a spectral signal, and the analysis value is a standard deviation. The signal analyzing step further comprises a step: analyzing the spectral signal and the interference signal to work out a DC term (representing the background light) and deduct the DC term to obtain the standard deviation of a cross-correlation term (representing the signal light).

In the comparing step, the target analysis value is a maximum one of the plurality of standard deviations.

Thereby, the autofocus method of this embodiment is applicable to an OCT image system using a low order computing device, accelerating the analysis and comparison process, reducing the computation time of autofocusing, and achieving a focusing precision as well as the conventional technology.

In order to achieve the abovementioned objectives, the present invention also provides an optical coherence tomography (OCT) image system, which is used to execute the abovementioned autofocus method, whereby to obtain OCT images of eyeballs. The OCT image system comprises a light emitter, an optical assembly, a focusing driving device, a control processor, and an image output device.

The light emitter emits a light beam, and the optical assembly is optically coupled to the light beam.

The optical assembly further comprises a splitter, a collimator assembly, and a light signal receiver. The splitter receives the light beam and splits the light beam into a sampling light beam and a reference light beam. The collimator assembly has collimators respectively at the side of the sampling light beam and the side of the reference light beam. The sampling light beam is projected onto an eyeball and then reflected to the collimator. The reference light beam is projected onto a reference mirror and then reflected to the collimator. The splitter merges the sampling light beam and the reference light beam to form an interference signal. The light signal receiver receives the interference signal. The focusing driving device is arranged inside the optical assembly and used to adjust the diopter at which the sampling light beam is projected onto the eyeball. The control processor is coupled to the focusing driving device.

The focusing driving device is moved within the preset diopter range to reach one of a plurality of preset diopter positions of the preset diopter range. The control processor captures the reference charts at the plurality of preset diopter positions and acquires the interference signals from the light signal receiver. The control processor analyzes the plurality of reference charts and the corresponding interference signals thereof to obtain the analysis values and the corresponding diopters. The control processor compares the plurality of analysis values to obtain from the plurality of analysis values one analysis value meeting a standard condition as a target analysis value. According to the target analysis value and the corresponding diopter, the control processor controls the focusing driving device to move to the corresponding diopter position. Thus, autofocusing is completed.

The image output device is coupled to the control processor. According to the target focusing distance obtained after focusing is completed, the image output device outputs a corresponding reference chart, which represents the post-focusing OCT image.

In order to achieve the abovementioned objectives, the present invention further provides an optical coherence tomography (OCT) image system, which can reduce computation load, and which can perform autofocusing to obtain OCT images of eyeballs. The OCT image system comprises a storage device, a fundus camera, a light emitter, an optical assembly, an autofocusing device, a control processor, and an image output device. The storage device stores a reference focal length table. The reference focal length table includes a plurality of reference fundus-focused focal lengths and reference focal lengths corresponding to the reference fundus-focused focal lengths. The fundus camera performs autofocusing to obtain a current fundus-focused focal distance.

The light emitter emits a light beam, and the optical assembly is optically coupled to the light beam. The optical assembly further comprises a splitter, a collimator assembly, a reference mirror, and a light signal receiver. The splitter receives the light beam and splits the light beam into a sampling light beam and a reference light beam. The collimator assembly has collimators respectively at the side of the sampling light beam and the side of the reference light beam. The sampling light beam is projected onto an eyeball and then reflected to the collimator. The reference light beam is projected onto the reference mirror and then reflected to the collimator. The splitter merges the sampling light beam and the reference light beam to form an interference signal. The light signal receiver receives the interference signal. The focusing driving device is arranged inside the optical assembly and used to adjust the diopter at which the sampling light beam is projected onto the eyeball.

The control processor is coupled to the storage device and the focusing driving device. According to the current fundus-focused focal distance, the control processor looks up the reference focal length table to find out a reference fundus-focused focal length matching the current fundus-focused focal distance and uses a reference focal length, which is corresponding to the matched reference fundus-focused focal length, as a target focusing distance. The control processor controls the focusing driving device to move to the target focusing distance within the preset diopter range. Thus, autofocusing is completed.

The image output device is coupled to the control processor. According to the target focusing distance obtained after focusing is completed, the image output device outputs a corresponding reference chart, which represents the post-focusing OCT image.

In order to achieve the abovementioned objectives, the present invention further provides an autofocus method, which can reduce computation load, and which is applied to the abovementioned OCT image system. A reference focal length table is established for the OCT image system within the preset diopter range. The reference focal length table includes a plurality of reference fundus-focused focal lengths and reference focal lengths corresponding to the reference fundus-focused focal lengths.

The autofocus method comprises steps: a fundus focusing step: using the fundus camera to obtain a current fundus-focused focal distance; a comparing step: looking up a reference focal length table according to the current fundus-focused focal distance to find out a reference fundus-focused focal length matching the current fundus-focused focal distance and using a reference focal length, which is corresponding to the matched reference fundus-focused focal length, as a target focusing distance; and a focusing step: controlling a focusing driving device to move to the target focusing distance within the preset diopter range to complete autofocusing.

In comparison with the conventional technology, the OCT image system and the autofocus method of the present invention may reduce the computation load of analysis and conversion of signals, use a lower order computing device for cost reduction, decrease shooting time, increase shooting success rate, and provide better user experience.

The objectives, technologies, features and advantages of the present invention will become apparent from the following description in conjunction with the accompanying drawings wherein certain embodiments of the present invention are set forth by way of illustration and example.

### BRIEF DESCRIPTION OF THE DRAWINGS

The foregoing conceptions and their accompanying advantages of this invention will become more readily appreciated after being better understood by referring to the following detailed description, in conjunction with the accompanying drawings, wherein:

FIG. 1 is a flowchart of an autofocus method according to one embodiment of the present invention;

FIG. 2 is a block diagram schematically showing an optical coherence tomography image system according to a first embodiment of the present invention;

FIG. 3 is a flowchart of an autofocus method according to the first embodiment of the present invention;

FIG. 4 is a block diagram schematically showing an optical coherence tomography image system according to a second embodiment of the present invention;

FIG. 5 is a flowchart of an autofocus method according to the second embodiment of the present invention;

FIG. 6 is a diagram showing ocular images respectively captured at a plurality of preset diopter positions;

FIG. 7 is a diagram for comparing the ocular images respectively captured by the present invention and the conventional technology;

FIG. 8 is a block diagram schematically showing an optical coherence tomography image system according to a third embodiment of the present invention; and

FIG. 9 is a flowchart of an autofocus method according to the third embodiment of the present invention.

### DESCRIPTION OF THE PREFERRED EMBODIMENTS

Various embodiments of the present invention will be described in detail below and illustrated in conjunction with the accompanying drawings. In addition to these detailed descriptions, the present invention can be widely implemented in other embodiments, and apparent alternations, modifications and equivalent changes of any mentioned embodiments are all included within the scope of the present invention and based on the scope of the Claims. In the descriptions of the specification, in order to make readers have a more complete understanding about the present invention, many specific details are provided; however, the present invention may be implemented without parts of or all the specific details. In addition, the well-known steps or elements are not described in detail, in order to avoid unnecessary limitations to the present invention. Same or similar elements in Figures will be indicated by same or similar reference numbers. It is noted that the Figures are schematic and may not represent the actual size or number of the elements. For clearness of the Figures, some details may not be fully depicted.

The following text and the related drawings will be used to further demonstrate the embodiments of the present invention. The identical symbol will be use to designate the similar or identical component in the specification and drawings as far as possible.

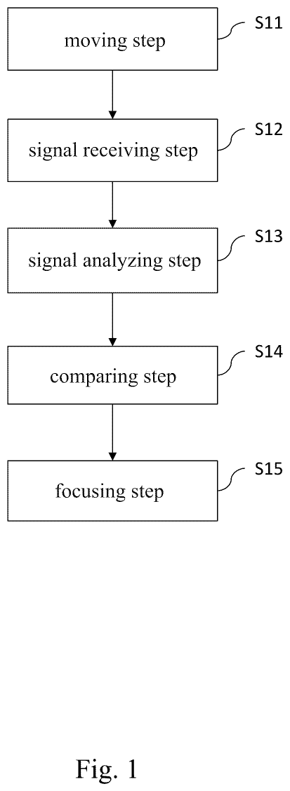

Refer to FIG. 1. The autofocus method of the present invention comprises a moving step (Step S11), a signal receiving step (Step S12), a signal analyzing step (Step S13), a comparing step (Step S14), and a focusing step (Step S15).

In the moving step (Step S11), move a focusing driving device within a preset diopter range to reach one of a plurality of preset diopter positions.

In the signal receiving step (Step S12), capture reference charts at the plurality of preset diopter positions and receive an interference signal generated by the reflection of a light beam projected on an eyeball.

Repeat Step S11 and Step S12 until the corresponding reference charts and interference signals have been obtained at all the preset diopter positions.

In the signal analyzing step (Step S13), analyze the reference charts and the corresponding interference signals to obtain a plurality of analysis values and the corresponding diopters.

In the comparing step (Step S14), analyze the plurality of analysis values to obtain from the plurality of analysis values one analysis value meeting a standard condition as a target analysis value.

In the focusing step (Step S15), according to the target analysis value and the corresponding diopter, control a focusing driving device to move to the corresponding diopter position, whereby autofocusing is completed.

In the moving step (Step S11), the preset diopter range is from −15D to 15D; there are 16 preset diopter positions within the preset diopter range from −15D to 15D; each two adjacent diopter positions are separated by 2D. In other words, the preset diopter positions include −15D, −13D, −11D . . . 13D, and 15D. The diopter D is equal to the reciprocal of the focal length f.

In the signal receiving step (Step S12), the reference charts are respectively captured at the abovementioned 16 preset diopter positions to facilitate undertaking Steps 13-15.

Thereby, the autofocus method of the present invention performs autofocusing without using complicated computation and uses less time in autofocusing than the conventional OCT device.

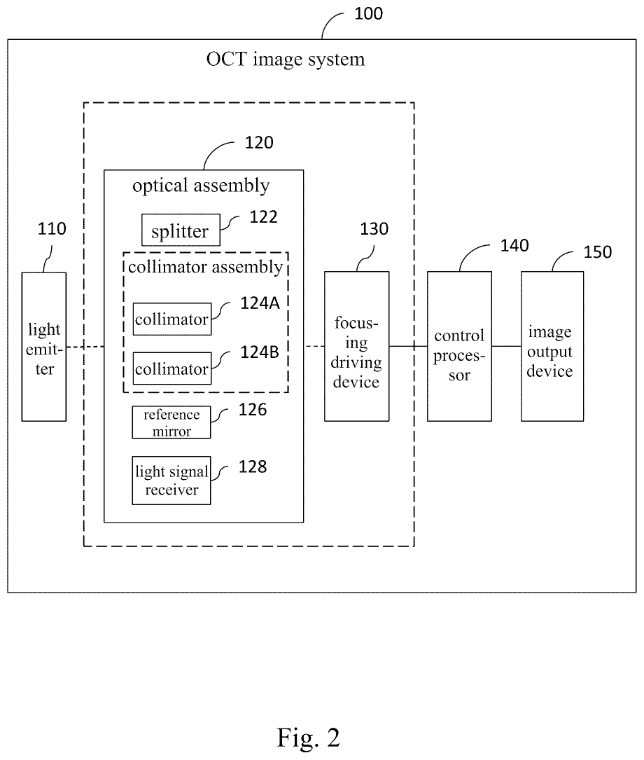

Refer to FIG. 2. According to a first embodiment, the OCT image system 100 of the present invention comprises a light emitter 110, an optical assembly 120, a focusing driving device 130, a control processor 140, and an image output device 150.

The light emitter 110 emits a light beam. The light emitter 110 may be a superluminescent diodes (SLD), a supercontinuum laser, or a swept source laser.

The optical assembly 120 further comprises a splitter 122, a collimator assembly, a reference mirror 126, and a light signal receiver 128. The splitter 122 receives the light beam and splits the light beam into a sampling light beam and a reference light beam. The collimator assembly has collimators 124A and 124B respectively at the side of the sampling light beam and the side of the reference light beam. The sampling light beam is projected onto an eyeball and then reflected to the collimator 124A. The reference light beam is projected onto the reference mirror 126 and then reflected to the collimator 124B. The splitter 122 merges the sampling light beam and the reference light beam to form an interference signal. The light signal receiver 128 receives the interference signal.

The focusing driving device 130 is arranged inside the optical assembly 120 and used to adjust the diopter at which the sampling light beam is projected onto the eyeball. The focusing driving device 130 may be a tunable lens or a combination of a motor and a lens, wherein the motor is used to drive the lens to move.

The control processor 140 is coupled to the focusing driving device 130. The control processor 140 controls the focusing driving device 130 to move within the preset diopter range to reach one of a plurality of preset diopter positions of the preset diopter range. The control processor 140 captures reference charts at the plurality of preset diopter positions and acquires interference signals of the light signal receiver 128. The control processor 140 analyzes the plurality of reference charts and the corresponding interference signals to obtain the analysis values and the corresponding diopters. The control processor compares the plurality of analysis values to obtain from the plurality of analysis values one analysis value meeting a standard condition as a target analysis value. According to the target analysis value and the corresponding diopter, the control processor 140 controls the focusing driving device 130 to move to the corresponding diopter position. Thus, autofocusing is completed.

The image output device 150 is coupled to the control processor 140. According to the diopter position obtained after focusing is completed, the image output device 150 outputs a corresponding reference chart, which represents the post-focusing OCT image.

The focusing driving device 130 further comprises an optical path adjuster and a focal length regulator. The optical path adjuster is disposed in the path of the reference light beam and used to adjust the optical path of the reference light beam. The focal length regulator is disposed in the path of the sampling light beam and used to adjust the focal length between the sampling light beam and the eyeball.

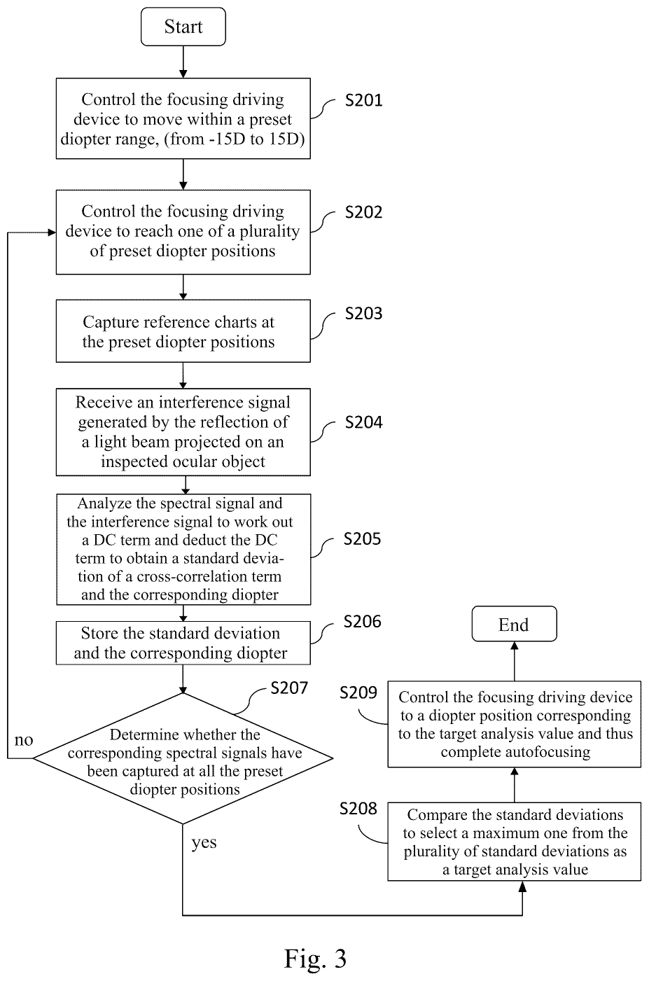

Refer to FIG. 2 and FIG. 3 simultaneously. FIG. 3 shows an autofocus method according to the first embodiment of the present invention. The autofocus method is applicable to the OCT image system. The autofocus method comprises steps:

Step S201: control the focusing driving device 130 to move within a preset diopter range, such a diopter range from −15D to 15D.

Step S202: the control processor 140 controls the focusing driving device 130 to reach one of a plurality of preset diopter positions.

Step S203: capture reference charts at the preset diopter positions.

Step S204: receive an interference signal generated by the reflection of a light beam projected on an eyeball.

Step S205: analyze the spectral signal and the interference signal to work out a DC term (representing the background light) and deduct the DC term to obtain a standard deviation of a cross-correlation term (representing the signal light) and the corresponding diopter.

Step S206: store the standard deviation and the corresponding diopter.

Step S207: determine whether the corresponding spectral signals have been captured at all the preset diopter positions.

Step S208: compare the plurality of standard deviations to select a maximum one from the plurality of standard deviations as a target analysis value.

Step S209: control the focusing driving device 130 to a diopter position corresponding to the target analysis value and thus complete autofocusing.

In this embodiment, the light signal receiver 128 may be a spectrometer used to receive spectral signals.

Therefore, the autofocus method of the present invention can reduce complicated computation and overcome the problems of the conventional technology, wherein the conventional technology suffers too long an overall computation time because of needing to capture the tomography images and perform computations (including the Fourier Transform) for focusing. Therefore, the present invention can raise the computation speed, decrease the computation cost, and achieve fast and accurate focusing.

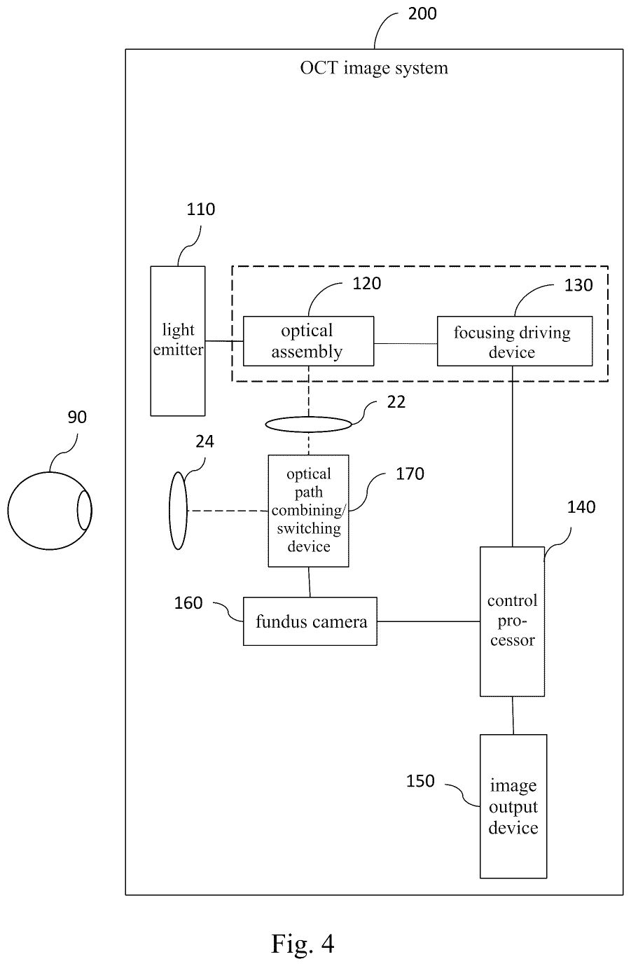

FIG. 4 schematically an OCT image system 200 according to a second embodiment of the present invention. The OCT image system 200 is different from the OCT image system 100 of the first embodiment in that the OCT image system 200 possesses a fundus camera and an optical path combining/switching device 170. The other part of the OCT image system 200 is the same as the OCT image system 100 and will not repeat herein.

In the optical assembly 120, the sampling light beam enters the collimator; the focusing driving device adjusts the focal length; then the sampling light beam is projected onto an eyeball 90 through a scanning lens 22 and an ocular lens 24. Between the scanning lens 22 and the ocular lens 24, the optical path combining/switching device 170 (such as a beam splitter or a dichroic mirror) is combined with the fundus camera 160 to capture the ocular image of the eyeball 90.

The optical assembly 120 may include a light signal receiver. The light signal receiver may be a photodetector or a balanced photodetector. The focusing driving device 130 may include a diopter regulator. The diopter regulator is used to regulate the diopter of the sampling light beam to make the focal surface of the sampling light beam focused at different depths of the eyeball 90. Then, the autofocus method automatically focuses the sampling light beam on the eyeball 90.

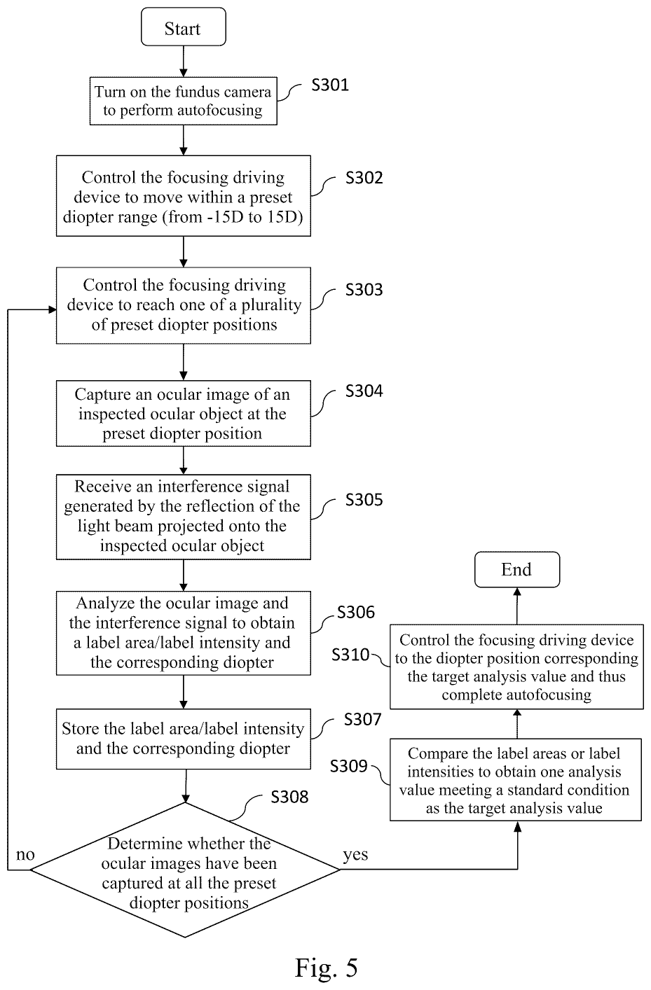

Refer to FIG. 4 and FIG. 5 simultaneously. FIG. 5 shows an autofocus method according to the second embodiment of the present invention. The autofocus method is applicable to the OCT image system 200 having the fundus camera 160. The autofocus method of the second embodiment comprises steps:

Step S301: turn on the fundus camera 160 to perform autofocusing.

Step S302: the control processor 140 controls the focusing driving device 130 to move within a preset diopter range, such a diopter range from −15D to 15D.

Step S303: control the focusing driving device 130 to reach one of a plurality of preset diopter positions.

Step S304: use the control processor 140 to control the fundus camera 160 to capture ocular images of the eyeball 90 at the preset diopter position.

Step S305: use the optical assembly 120 to receive an interference signal generated by the reflection of the light beam projected onto the eyeball 90.

Step S306: use the control processor 140 to analyze the ocular image of the eyeball 90 and the interference signal received by the optical assembly 120 to obtain a mark area/mark intensity and the corresponding diopter.

Step S307: store the mark area/mark intensity and the corresponding diopter.

Step S308: determine whether the ocular images have been captured at all the preset diopter positions. If no, the process returns to Step S303 to move the focusing driving device 130 once again. If yes, the process proceeds to Step S309.

Step S309: compare the mark areas or mark intensities to obtain one analysis value meeting a standard condition as the target analysis value.

Step S310: control the focusing driving device to the diopter position corresponding to the target analysis value and thus complete autofocusing.

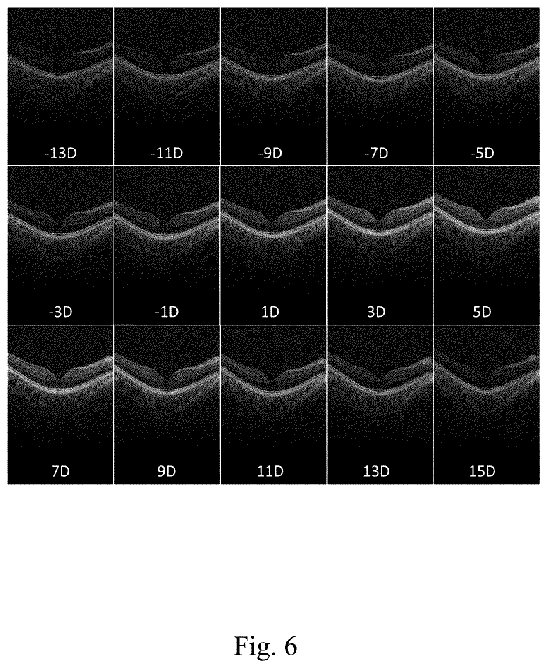

Refer to FIG. 5 and FIG. 6 simultaneously. In Step S303, 16 preset diopter positions are established within the preset diopter range from −15D to 15D, and two adjacent preset diopter positions are separated by 2D. In Step S303, the focusing driving device is controlled to reach the preset diopter position of −15D of the preset diopter range from −15D to 15D, and then Step S304-Step S307 are executed; in the next cycle, the focusing driving device is controlled to reach the preset diopter position of −13D, and then Step S304-Step S307 are executed. The similar cycles are repeated until it is determined that the ocular images have been captured at all the 16 preset diopter positions within the preset diopter range from −15D to 15D in Step S308. Then is undertaken Step S309.

In Step S309, compare the 16 mark areas/mark intensities, which are obtained in Step S306 and stored in Step S307.

If the standard condition is set to be the minimum one of the plurality of mark areas, find out the minimum one of the 16 mark areas. If the mark area acquired at the diopter position of 5D is the minimum one, the focusing driving device is moved to the position corresponding to the diopter of 5D in Step S310 to complete autofocusing.

If the standard condition is set to be the maximum one of the plurality of mark intensities, find out the maximum one of the 16 mark intensities. If the mark intensity acquired at the diopter position of 5D is the maximum one, the focusing driving device is moved to the position corresponding to the diopter of 5D in Step S310 to complete autofocusing.

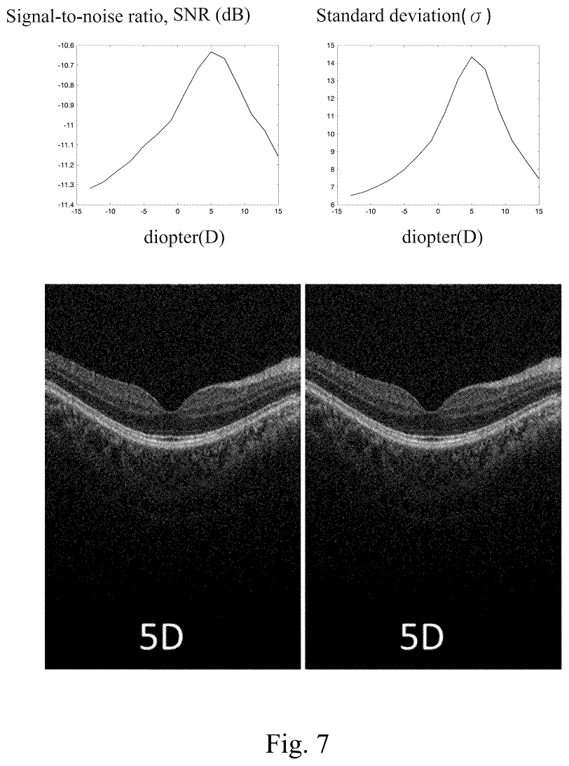

Refer to FIG. 7. FIG. 7 schematically shows the ocular images respectively obtained by the present invention and the conventional technology. The left side of FIG. 7 shows the ocular mage obtained by the conventional SNR autofocus method based on calculating SNR. The right side of FIG. 7 shows the ocular mage obtained by the autofocus method of the present invention. FIG. 7 proves that the autofocusing result output by present invention is highly similar to that output by the conventional technology. However, it should be noted: the present invention is exempted from complicated Fourier Transform and other computation; the overall autofocusing time is reduced from 1.31 seconds to 0.25 seconds. In other words, the autofocusing speed is raised 5 times.

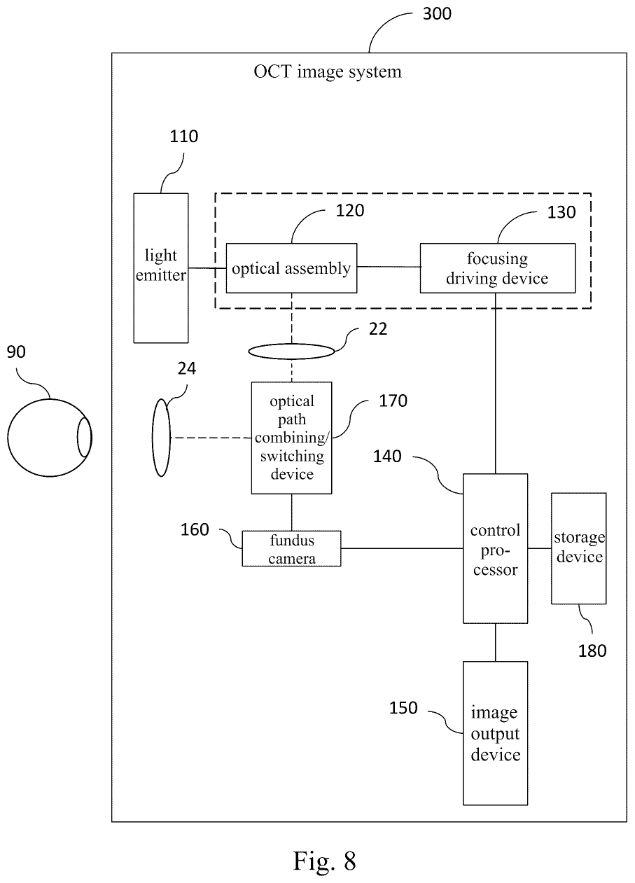

FIG. 8 schematically an OCT image system 300 according to a third embodiment of the present invention. The OCT image system 300 is different from the OCT image system 100 of the first embodiment in that the OCT image system 300 possesses a storage device 180.

The storage device 180 stores a reference focal length table. The reference focal length table includes a plurality of reference fundus-focused focal lengths and a plurality of reference focused lengths corresponding to the reference fundus-focused focal lengths.

TABLE 1
Reference Focal Length

| Reference Fundus- | |
|---|---|
| Focused Focal Length | Reference Focal Length |
| f₁₁ | f₂₁ |
| f₁₂ | f₂₂ |
| f₁₃ | f₂₃ |
| f₁₄ | f₂₄ |
| f₁₅ | f₂₅ |

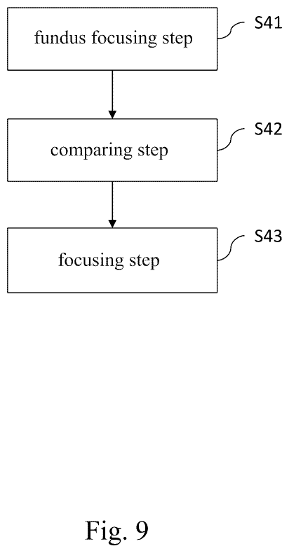

Refer to FIG. 8 and FIG. 9 simultaneously. FIG. 9 is a flowchart of an autofocus method according to the third embodiment of the present invention. The autofocus method of the third embodiment comprises a fundus focusing step (Step S41), a comparing step (Step S42), and a focusing step (Step S43).

In the fundus focusing step (Step S41), use the fundus camera 160 to obtain a current fundus-focused focal distance.

In the comparing step (Step S42), the control processor 140 looks up the reference focal length table according to the current fundus-focused focal distance to find out a reference fundus-focused focal length matching the current fundus-focused focal distance and uses the reference focal length, which is corresponding to the matched reference fundus-focused focal length, as a target focusing distance.

In the focusing step (Step S43), the control processor 140 controls the focusing driving device 130 to move to the target focusing distance within the preset diopter range and thus completes autofocusing.

For example, the current fundus-focused focal distance is acquired in Step S41 beforehand. Suppose that the current fundus-focused focal distance is f14. Then, in Step S42, look up Table. 1 (listing the reference focal lengths) to find out the reference fundus-focused focal length f14 matching the current fundus-focused focal distance f14, and used a reference focal length f24, which is corresponding to the matched reference fundus-focused focal length f14, as a target focusing distance f24. Thus, the target focusing distance f24 is found out in Step S42. Then, instep S43, the control processor 140 controls the focusing driving device 130 to move to the target focusing distance f24 to complete autofocusing.

Thereby, the present provides an OCT image system and an autofocus method thereof, which can raise the shooting success rate, reduce fabrication cost, and decrease computation load of signal analysis, and which can still output high-quality OCT images using a lower-order computing device.

While the invention is susceptible to various modifications and alternative forms, a specific example thereof has been shown in the drawings and is herein described in detail. It should be understood, however, that the invention is not to be limited to the particular form disclosed, but to the contrary, the invention is to cover all modifications, equivalents, and alternatives falling within the appended claims.

1. An autofocus method, which is applied to an optical coherence tomography image system, comprising steps:
turning on an autofocusing function of a fundus camera and letting the autofocusing function of the fundus camera be completed;
undertaking a moving step: controlling a focusing driving device to move within a preset diopter range to reach one of a plurality of preset diopter positions of the preset diopter range;
undertaking a signal receiving step: capturing a reference chart at the plurality of preset diopter positions, wherein the reference chart is an ocular image of an eyeball, and receiving an interference signal generated by reflection of a light beam projected on the eyeball, and controlling the fundus camera to capture the ocular images at the plurality of preset diopter positions;
undertaking the moving step and the signal receiving step repeatedly to respectively obtain the ocular images and the interference signals at the plurality of preset diopter positions;
undertaking a signal analyzing step: analyzing the ocular images and the interference signals to obtain a plurality of corresponding analysis values and corresponding diopters, wherein the plurality of analysis values is a plurality of mark areas or mark intensities; analyzing a light source mark in the ocular image to obtain the mark area or mark intensity of the light source mark;
undertaking a comparing step: analyzing the plurality of analysis values and obtaining from the plurality of analysis values one analysis value meeting a standard condition as a target analysis value; and
undertaking a focusing step: according to the target analysis value and the corresponding diopter, controlling the focusing driving device to move to a position corresponding to the diopter to complete autofocusing.

2. The autofocus method according to claim 1, wherein in the comparing step, the standard condition is a minimum one of the plurality of mark areas.

3. The autofocus method according to claim 1, wherein in the comparing step, the standard condition is a maximum one of the plurality of mark intensities.

4. An autofocus method, which is applied to an optical coherence tomography image system, comprising steps:
undertaking a moving step: controlling a focusing driving device to move within a preset diopter range to reach one of a plurality of preset diopter positions of the preset diopter range;
undertaking a signal receiving step: capturing a reference chart at the plurality of preset diopter positions, and receiving an interference signal generated by reflection of a light beam projected on an eyeball;
undertaking the moving step and the signal receiving step repeatedly to respectively obtain the ocular images and the interference signals at the plurality of preset diopter positions;
undertaking a signal analyzing step: analyzing the reference chart and the interference signal to obtain an analysis value and a corresponding diopter, wherein the reference chart is a spectral signal, and the analysis value is a standard deviation;
analyzing the spectral signal and the interference signal to work out a DC term and deduct the DC term to obtain a standard deviation of a cross-correlation term and a diopter corresponding to the standard deviation;
undertaking a comparing step: comparing the plurality of standard deviations to select from the plurality of standard deviations one standard deviation meeting a standard condition as a target analysis value; and
undertaking a focusing step: according to the target analysis value and the corresponding diopter, controlling the focusing driving device to move to a position corresponding to the diopter to complete autofocusing.

5. The autofocus method according to claim 4, wherein in the comparing step, the standard condition is a minimum one of the plurality of deviations.

6. An optical coherence tomography image system, which is applied to the autofocus method of claim 1 for obtaining a tomography image of an eyeball, comprising
a light emitter, emitting a light beam;
an optical assembly, optically coupled to the light beam, and further comprising a splitter, a collimator assembly, a reference mirror, and a light signal receiver, wherein the splitter receives the light beam and splits the light beam into a sampling light beam and a reference light beam; the collimator assembly has collimators respectively at a side of the sampling light beam and a side of the reference light beam; the sampling light beam is projected onto an eyeball and then reflected to the collimator; the reference light beam is projected onto the reference mirror and then reflected to the collimator; the splitter merges the sampling light beam and the reference light beam to form an interference signal; the light signal receiver receives the interference signal;
a focusing driving device, arranged inside the optical assembly and used to adjust a diopter at which the sampling light beam is projected onto the eyeball;
a control processor, coupled to the focusing driving device, controlling the focusing driving device to move within a preset diopter range to reach one of a plurality of preset diopter positions of the preset diopter range, respectively capturing reference charts at the plurality of preset diopter positions and acquiring the interference signal from the light signal receiver, wherein the reference chart is an ocular image, analyzing a light source mark of the light beam in the plurality of ocular images to obtain a mark area or mark intensity of the light source mark, wherein the analysis value is a mark area or a mark intensity; comparing the plurality of analysis values to obtain from the plurality of analysis values one analysis value meeting a standard condition as a target analysis value; according to the target analysis value and the corresponding diopter, controlling the focusing driving device to move to the corresponding diopter position to complete autofocusing; and
an image output device, coupled to the control processor; according to the diopter position obtained in focusing, outputting the ocular image corresponding to the diopter, which represents a post-focusing tomography image.

7. The optical coherence tomography image system according to claim 6, further comprising
a fundus camera, coupled to the control processor, and performing an autofocusing function to capture an ocular image of the eyeball.

8. The optical coherence tomography image system according to claim 7, wherein the control processor controls the fundus camera to capture the ocular images at the plurality of preset diopter positions.

9. The optical coherence tomography image system according to claim 7, wherein the fundus camera is optically coupled to the optical assembly and used to capture an ocular image having a pupil and a fundus.

10. The optical coherence tomography image system according to claim 6, wherein the standard condition is a minimum one of the plurality of mark areas.

11. The optical coherence tomography image system according to claim 6, wherein the standard condition is a maximum one of the plurality of mark intensities.

12. The optical coherence tomography image system according to claim 6, wherein the light emitter is a superluminescent diode (SLD), a supercontinuum laser, or a swept source laser.

13. The optical coherence tomography image system according to claim 6, wherein the light signal receiver is a photodetector, a balanced photodetector, or a spectrometer.

14. The optical coherence tomography image system according to claim 6, wherein the focusing driving device includes a motor and a lens, and the motor is used to drive the lens to move.

15. The optical coherence tomography image system according to claim 6, wherein the focusing driving device includes a tunable lens.

16. An optical coherence tomography image system, which is used to execute the autofocus method of claim 4 for obtaining a tomography image of an eyeball, comprising
a light emitter, emitting a light beam;
an optical assembly, optically coupled to the light beam, and further comprising a splitter, a collimator assembly, a reference mirror, and a light signal receiver, wherein the splitter receives the light beam and splits the light beam into a sampling light beam and a reference light beam; the collimator assembly has collimators respectively at a side of the sampling light beam and a side of the reference light beam; the sampling light beam is projected onto an eyeball and then reflected to the collimator; the reference light beam is projected onto the reference mirror and then reflected to the collimator; the splitter merges the sampling light beam and the reference light beam to form an interference signal; the light signal receiver receives the interference signal;
a focusing driving device, arranged inside the optical assembly and used to adjust a diopter at which the sampling light beam is projected onto the eyeball;
a control processor, coupled to the focusing driving device, controlling the focusing driving device to move within a preset diopter range to reach one of a plurality of preset diopter positions of the preset diopter range, respectively capturing reference charts at the plurality of preset diopter positions and acquiring the interference signal from the light signal receiver, wherein the reference chart is a spectral signal, analyzing the plurality of spectral signals and the corresponding interference signals to obtain an analysis value and the corresponding diopter, wherein the analysis value is a standard deviation; comparing the plurality of standard deviations to obtain from the plurality of standard deviations one standard deviation meeting a standard condition as a target analysis value; according to the target analysis value and the corresponding diopter, controlling the focusing driving device to move to the corresponding diopter position to complete autofocusing; analyzing the spectral signal and the interference signal to work out a DC term and deduct the DC term to obtain the standard deviation of a cross-correlation term; and
an image output device, coupled to the control processor; according to the diopter position obtained in focusing, outputting the reference chart corresponding to the diopter, which represents a post-focusing tomography image.

17. The optical coherence tomography image system according to claim 16, wherein the standard condition is a maximum one of the plurality of standard deviations.

18. The optical coherence tomography image system according to claim 16, wherein the light emitter is a superluminescent diode (SLD), a supercontinuum laser, or a swept source laser.

19. The optical coherence tomography image system according to claim 16, wherein the light signal receiver is a photodetector, a balanced photodetector, or a spectrometer.

20. The optical coherence tomography image system according to claim 16, wherein the focusing driving device includes a motor and a lens, and the motor is used to drive the lens to move.

21. The optical coherence tomography image system according to claim 16, wherein the focusing driving device includes a tunable lens.
